(19) United States
(12) Patent Application Publication
(10) Pub. No.: US 2026/0276501 A1
(43) Pub. Date: Sep. 17, 2026

(54) SYSTEMS AND METHODS FOR DETERMINING COMPRESSION PROPERTIES OF A POWDER AND USEFUL, FOR EXAMPLE, WITH TABLET FORMULATION DEVELOPMENT

(71) Applicant: Regents of the University of Minnesota, Minneapolis, MN (US)

(72) Inventors: Changquan Calvin Sun, Minneapolis, MN (US); Gerrit Vreeman, Fairbanks, AK (US)

(73) Assignee: Regents of the University of Minnesota, Minneapolis, MN (US)

(21) Appl. No.: 19/471,242

(22) PCT Filed: Mar. 29, 2024
(86) PCT No.: PCT/US2024/022383

§ 371 Date: Sep. 30, 2025

Related U.S. Application Data
(60) Provisional application No. 63/456,848, filed on Apr. 4, 2023.

(51) Int. Cl. G01N 3/08; G01N 33/00

(52) U.S. Cl. CPC G01N 3/08; G01N 33/0091; G01N 2203/0019; G01N 2203/0085; G01N 2203/0087; G01N 2203/0218; G01N 2203/0252; G01N 2203/0284

(57) ABSTRACT

Systems and methods for analyzing powder compaction data to extract or derive parameters of the powder. With methods of the present disclosure, in-die compaction simulator data is collected and analyzed, and requires only a relatively small quantity of material. The systems and methods can utilize an additive mathematical model of powder compressibility during uniaxial compaction by combining bulk elasticity, linear and non-linear, and low-to-medium pressure densification processes for assessing a powder material under consideration.

### BACKGROUND

The present disclosure relates to evaluation of powder materials. More particularly, it relates to deriving properties or parameters of a powder material based on data obtained during uniaxial compaction.

Powder compaction plays an important role in many industries, including pharmaceuticals, ceramics, metallurgy, pellet fuels, cosmetics, foods, detergents, and soil sciences. For example, successful tableting of active pharmaceutical ingredient (API) powders requires an understanding and analysis of fundamental properties or parameters of the powders. By understanding the effect of important parameters during compaction, some difficulties in tableting can be avoided or solved.

To fully understand the properties of pharmaceutical powder important for tablet development, a few techniques are currently available. Instruments required for these existing studies are expensive and require a substantial quantity of the material to be evaluated (e.g., grams to tens of grams). Additionally, expertise in each of these techniques is needed for data interpretation and accuracy of data. Some existing techniques are sensitive to environmental and experimental factors that are hard to control (e.g., humidity).

### SUMMARY

The inventors of the present disclosure recognized that a need exists for improved systems and methods for determining the properties of a powder material.

Some aspects of the present disclosure relate to systems and methods for analyzing powder compaction data to extract or derive parameters of the powder. In some embodiments, the systems and methods of the present disclosure review compaction data generated in-die by a compaction simulator, and require only a relatively small quantity of material (e.g., on the order of less than one gram). The systems and methods of the present disclosure can utilize an additive mathematical model of powder compressibility during uniaxial compaction by combining bulk elasticity, linear and non-linear, and low-to-medium pressure densification processes for assessing a powder material under consideration. The flexibility of this model allows for an adequate description of several densification mechanisms, including fragmentation, rearrangement, and plastic deformation. Out-of-die parameters can be estimated from in-die compressibility data by deconvoluting the bulk elastic contribution to volume reduction under pressure. A number of useful parameters may be extracted from such an analysis, such as true density (ρₜ), mean yield pressure (P_{y}), and constrained modulus (M). Out-of-die compressibility may be considered a special case of some of the models of the present disclosure. The derived parameter(s) provided by the systems and methods of the present disclosure can be used for screening solid form of an API with superior mechanical properties early in drug development, or profiling batch-to-batch variation of an API efficiently. Additionally, accurately modeling compressibility by such a materials- and time-sparing approach is beneficial in other fields, such as detergent manufacturing, soil compaction, and powder metallurgy.

### BRIEF DESCRIPTION OF THE DRAWINGS

FIG. 1 is a block diagram of an evaluation system in accordance with principles of the present disclosure;

FIG. 2 is an example Heckel plot:

FIG. 3A is a plot of porosity as a function of applied pressure for an example material subjected to compression according to the additive models of the present disclosure;

FIG. 3B is an example plot of density as a function of applied pressure for the example material subjected to compression of FIG. 3A and according to the additive models of the present disclosure;

FIG. 3C is an example plot of volume as a function of applied pressure for the example material subjected to compression of FIG. 3A and according to the additive models of the present disclosure;

FIGS. 4A and 4B are graphs reporting fitting compression data and residuals described in the Examples section:

FIGS. 5A and 5B are graphs reporting fitting compression data and residuals described in the Examples section;

FIG. 6 illustrates a compression and decompression curve for a material, microcrystalline cellulose, described in the Examples section; and

FIG. 7 is a graph reporting in- and out-of-die density versus compaction pressure plots for a material, microcrystalline cellulose, described in the Examples section

### DETAILED DESCRIPTION

The present disclosure relates to systems and methods for the reliable characterization of material properties of powder materials using an in-die analysis. The in-die analyses of the present disclosure are fast and materials-sparing for assessing material compressibility.

One example of an evaluation system 20 in accordance with principles of the present disclosure, and useful for performing methods of the present disclosure, is shown in block form in FIG. 1. The system 20 includes a uniaxial compression device 30 and an assessment device 32. Details on the various components are provided below. In general terms, the compression device 30 is, or is akin to, an in-die uniaxial compression instrument or compaction simulator as is known in the art, generally configured to compress a powder between two punches in a die and dynamically measure or collected data during a compression operation, for example pressure (P), volume (V), density (ρ), and porosity (ε). The assessment device 32 is a computer or computer like device programmed (e.g., hardware and/or software) to derive a full range of compressibility information or parameters for the powder. In some embodiments, the assessment device 32 can receive collected data directly form the compression device 30 (e.g., via a wired or wireless connection). In other embodiments, data obtained during the operation of the compression device 30 is entered into the assessment device 32 by a user. Regardless, the assessment device 32 can be programmed to fit collected data utilizing the modeling or equations described below to obtain useful parameters and display or otherwise report the determined parameter(s) to a user.

The assessment device 32 is configured or programmed to produce quantitative evaluation data or information directly characterizing compressibility parameters of the powder subjected to testing by the compression device 30. The assessment device 32 includes a processor 50 coupled to one or more memories 52. The processor 50 can be a microprocessor, an embedded microprocessor, an embedded controller, a digital signal processor (DSP), etc. The processor 50 is configured to execute program code stored as software in the memory 52. The program code, when executed by the processor 50, causes the processor 50 to implement the assessment functions described herein. The processor 50 can reside in any suitable computing equipment, such as a personal computer, laptop, mobile electronic device, server or cloud-based computational platform. The processor 50 can further cooperate with the memory 52 to store data received from the compression device 30. In this regard, the assessment device 32 can include one or more features for receiving data from the compression device 30 either directly (e.g., wired or wireless interface with the compression device 30) or indirectly (e.g., sensor information collected during a test session can be separately stored and then loaded to the assessment device 32 for example via an optional input component 54 (for example a key board, touch screen, etc.)). The assessment device 32 can optionally include, or be connected to, additional components for conveying information to and/or receiving information from a user, such as a display 56.

The computer implemented methods of the present disclosure operate one or more algorithms or equations described below. In general terms, the models or algorithms of the present disclosure represent an additive model of powder compressibility during uniaxial compaction by combining bulk elasticity, linear or non-linear, and low-to-medium densification processes. By way of background, the compressibility of a powder is defined as the change in V of a powder bed in relation to the applied P. Compressibility can be equivalently described in terms of ρ, since V and ρ are inversely related as:

$$ρ = \frac{m}{V} \qquad Eq . ( 1 )$$

Where m is the powder mass. Similarly, compressibility can be described in terms of porosity (ε), which is a measure of the relative free space in the powder bed and determined by:

$$ε = 1 - \frac{ρ}{{ρ}_{t}} \qquad Eq . ( 2 )$$

Where ρₜ is the true density of the material, ρₜ is related to m and the true volume (Vₜ), which is the volume of a material excluding pores, according to Equation 3 as:

$${ρ}_{t} = \frac{m}{{V}_{t}} \qquad Eq . ( 3 )$$

Relative density (also known as solid fraction, defined as 1−ε), can also be used to describe compressibility. However, since it is intrinsically related to ε, no distinction is needed in terms of analyzing powder compressibility. Therefore, compressibility may be described using V, ρ, or ε as a function of P. It may be noted that when characterizing compressibility, p requires both V and m, while ε requires V, m, and ρₜ of the sample. Compressibility may also be determined as a function of punch distance as:

$$V = C ( c , A ) h + A * h \qquad Eq . ( 4 )$$

Where C(c,A) is a constant representing any concavity or convexity in the punch (c) and A is the punch surface area, and h is the distance between punches (that otherwise changes during compaction). Stated otherwise, h=(V−C(C,A))/A. In the case of a simple round, flat-faced tooling, C(c,A)=0 and A=πr², but is inherently a constant related to punch geometry. With this approach, h is geometry independent (and thus any punch geometry can be used).

As the pressure applied on a confined powder increases during the loading phase of powder compaction, multiple mechanisms contribute to a decrease in volume. Particle rearrangement occurs predominantly at low pressures, followed by particulate elastic deformation, fragmentation, plastic yield, and bulk elastic deformation at high pressures. In this description, “low”, “medium”, and “high” pressures are relative, depending on the material of interest. For example, a “high” pressure for a conventional rubber material will be lower than a “high” pressure for a standard ceramic (i.e., a much higher pressure is required to deform a ceramic than rubber). Thus, the value-agnostic description of low, medium, and high pressure is material-dependent.

When a powder is compressed uniaxially between two punches in a die, its compressibility can be studied using either an “out-of-die” or an “in-die” method. To collect “out-of-die” compressibility data, multiple compacts are formed at various pressures, ejected from the die, and subsequently characterized in terms of their V, ρ, or ε. To collect “in-die” compressibility data, the powder is compressed inside a die, and P, V, ρ, and ε are dynamically measured by the compressing instrument.

Out-of-die compressibility has been historically preferred since access to accurate in-die measurements was limited. Additionally, out-of-die compressibility is thought to be free from complicating factors, such as die-wall friction and particle elastic deformation, making data interpretation simpler. However, out-of-die compressibility has many drawbacks since it is time- and resource-intensive, requiring a few hours of data collection time and grams to tens of grams worth of materials. Additionally, out-of-die compressibility is influenced by errors in measured dimensions of a compact, which can be caused by tablet flashing. Furthermore, if intact compacts cannot be made due to capping, lamination, or other forms of defect upon ejection, out-of-die compressibility data cannot be obtained for analysis.

In contrast, in-die compressibility can be characterized using a single compression since the measurements are performed dynamically, making it time- and materials-sparing, requiring only a few seconds and tens to hundreds of milligrams to collect a set of data. Though influenced by die-wall friction and elasticity during compression, recent work has shown a strong correlation between in-die and out-of-die compressibility parameters, indicating its suitability for characterizing powders.

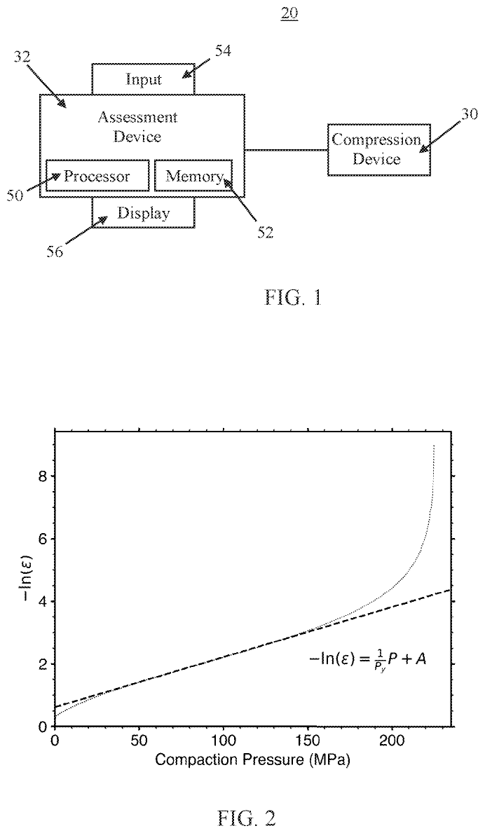

The most common mathematical model used to describe compressibility is the Heckel model, where the reduction in e with respect to an increase in P is assumed to be a first-order process (Equation 5 (also referred to as the “Heckel equation”) below). A negative log-transformed e plotted against P results in a characteristic compressibility curve. A typical Heckel plot is shown in FIG. 2. Regression to the linear portion of this curve reveals a plasticity parameter known as the mean yield pressure (P_{y}), which is related to the yield strength of the powder, and an empirical constant, A, which is thought to represent low-pressure packing.

$$- ln ( ε ) = \frac{1}{{P}_{y}} P + A \qquad Eq . ( 5 )$$

Materials with a lower P_{y} are considered more plastic since a lower pressure is required to result in plastic deformation. The main difference between the in-die and out-of-die Heckel data is the contribution of elastic deformation to in-die e, which artificially lowers the P_{y}. The out-of-die analysis is not influenced by elastic deformation, but is influenced by defects caused by excessive elastic recovery or air entrapment. Recently, it has been shown that P_{y} values obtained by properly performed in-die and out-of-die analyses follow a linear relationship, indicating that the in-die analysis is reliable despite the complication by elastic deformation. This, along with its many benefits (i.e., fast, materials sparing, simple), supports the use of in-die analyses to characterize powder plasticity. Despite its usefulness, the in-die Heckel analysis only describes the plasticity of the powder using a portion of the compressibility data. It does not describe the rearrangement and fragmentation that occurs at low pressures nor the bulk elastic deformation that occurs at high pressures. The choice of the linear portion of the curve may also vary between researchers, causing variability in the plasticity characterization for the same powder.

Gerdemann et al., “Compaction of Titanium Powders”, Metall. Mater. Trans. A 42, 1325-1333 (2011), the entire teachings of which are incorporated herein by reference, proposed Equation 6 (or “G-J equation”) below to describe the out-of-die compressibility of a number of titanium powders, which models compression data as an additive function between two independent first-order rate equations that account for the processes of rearrangement and work hardening.

$$ρ ( P ) = {ρ}_{0} + A * ( 1 - {e}^{- aP} ) + B * ( 1 - {e}^{- bP} ) \qquad Eq . ( 6 )$$

Where ρ₀ is the initial powder density, A and B are constants that quantify the densification extent, and a and b are related to the process half-lives of the two processes. The G-J equation (Equation 6) may be considered an extended version of the Heckel equation (Equation 5), which accounts for both the low-pressure densification processes and the medium-pressure plastic deformation process (where b=1/P_{y}). Describing compressibility as a function of ρ instead of ε is more beneficial since it is not affected by any errors in external pt measurements, and can be used to extract pt directly from compression data. For example, ρₜ may be difficult to unambiguously determine for water-containing powders by helium pycnometry. Fitting out-of-die compressibility data with Equation 5 allows for the elucidation of P_{y} without considering ε, which is required for applying the Heckel equation (Equation 5). More recently, an equivalent, albeit porosity-based, expression has been suggested to describe in-die compressibility of some pharmaceutically relevant materials after correcting for elastic deformation using a simplistic descriptor of material elasticity.

With the above background in mind and returning to FIG. 1, in some embodiments, the assessment device 32 operates a computer-implemented method for characterizing material properties using in-die data based on: 1) Each V reduction mechanism that occurs during powder compaction (e.g., rearrangement and fragmentation, plastic, and elastic deformation) can be individually mathematically described; and 2) The sum of each mechanism's mathematical representation describes the overall process of total V reduction as a function of P. Some examples of the bases for evaluations provided by the computer implemented methods of the present disclosure are provided below. In general terms, the assessment device 32 operates to model elastic deformation in accord with one or more of Equations 24-37 below, providing a reliable characterization of material properties using in-die data.

The flexible compressibility analyses provided by the systems and methods of the present disclosure account for multiple densification mechanisms, and yield significant material information that can directly inform downstream operations, such as formulation design and optimization, using a very small amount of material. For example, as previously discussed, accurate pt can be difficult to obtain using conventional methods or it takes a significant amount of time for accurate determination for some materials. Some embodiments of the present disclosure allow for the extraction of ρₜ using an in-die analysis of compressibility. Thus, an accurate ρₜ of an individual sample may be determined using just a small amount of powder (e.g., approx. 50-100 milligram (mg) for a 6.0 millimeter (mm) in diameter die) in only seconds. Therefore, ρₜ can be quickly determined, which makes it suitable for routine determination of batch-to-batch variations in ρₜ. Regardless, information generated by the assessment device 32 can be used with various additional methods of the present disclosure. For example, with some methods of the present disclosure, one or more modeled or fitted parameters (e.g., fragmentation; rearrangement; plastic deformation; nonlinear, linear, and zero elastic response; elastic properties; plastic properties; true density; out-of-die compressibility; etc.) as determined by the assessment device 32 via compaction data obtained or collected by the in-die compression device 30 during testing of a small sample of the material in question (e.g., on the order of less than 1 gram) can be used for screening solid form of an API with superior mechanical properties early in drug development, or batch-to-batch variation of an API may be efficiently profiled. Additionally, accurately modeling compressibility by such a materials- and time-sparing approach is beneficial in other fields, such as detergent manufacturing, soil and biocarbon compaction, and powder metallurgy.

Multiple processes occur during uniaxial compression of a confined powder, including elastic deformation (ε_{E}(P)), plastic yield (ε_{Y}(P)), and a series of other low-pressure mechanisms, such as particle rearrangement and fragmentation. These may be represented as a series of n mechanisms

$$( {Σ}_{i = 1}^{n} {ε}_{M , i} ( P ) ) .$$

Fragmentation may be negligible in some cases for particles below a material-specific critical size, e.g., most micronized APIs. In addition, rearrangement may also be negligible if particle packing is nearly ideal upon die filling. Other low-pressure mechanisms may be present depending on the powder. While these processes all occur during compaction, each dominates in a different pressure range. With some systems and methods of the present disclosure, volume elimination of a powder bed by each of these low- and medium-pressure mechanisms is assumed to follow first order kinetics, i.e., the rate of volume reduction by increasing pressure is proportional to the volume. Accordingly, Equation 7 below can describe compressibility over the entire pressure range.

$$ε ( P ) = {ε}_{E} ( P ) + {ε}_{Y} ( P ) + {\sum }_{i = 1}^{n} {ε}_{M , i} ( P ) \qquad Eq . ( 7 )$$

Bulk elastic deformation is predominant under pressures that lead to compact p that exceeds the material's ρₜ. At such pressures, the extent of contribution to further volume reduction from irreversible plastic deformation is negligible since there is little open space in the powder bed to accommodate plastic flow of particles. For a single powder compaction event, it can be assumed the process is isothermal. Hence, P can relate with V utilizing a material-dependent bulk modulus (K), which is related to the change in isostatic P with respect to a change in V using an equation of state (EOS) as follows:

$$K = - V \frac{d P}{d V} \qquad Eq . ( 8 )$$

The simplest isothermal EOS for solids assumes linear elasticity, i.e., K is independent of pressure. In this case, Equation 8 can be solved explicitly, resulting in Equation 9 below.

$$V ( P ) = {V}_{0} {e}^{- \frac{P}{K}} \qquad Eq . ( 9 )$$

Where V₀ in this instance is equivalent to the material's Vₜ. This can be rewritten in terms of ρ (Equation 10) and ε (Equation 11) below based on the definition of density (Equation 1) and porosity (Equation 2).

$$ρ ( P ) = {ρ}_{t} {e}^{\frac{P}{K}} \qquad Eq . ( 10 )$$

$$ε ( P ) = 1 - {e}^{\frac{P}{K}} \qquad Eq . ( 11 )$$

It has been observed that a material will become more difficult to compress as pressure increases. i.e., K is dependent on P, exhibiting a behavior known as non-linear elasticity. The simplest expression of non-linear elasticity is to consider K as a linear function of pressure, as given by the Murnaghan equation (Equation 12).

$$K = {K}_{0} + {PK}_{0}^{′} = - V \frac{d P}{d V} \qquad Eq . ( 12 )$$

Where K₀ is the initial bulk modulus at zero pressure and K₀′ is the first derivative of bulk modulus with respect to P. This separable differential equation can also be solved for V in terms of P as:

$$V ( P ) = {{V}_{0} [ 1 + P ( \frac{{K}_{0}^{′}}{{K}_{0}} ) ]}^{- \frac{1}{{K}_{0}^{′}}} \qquad Eq . ( 13 )$$

This expression can be converted into ε as a function of P as:

$$ε ( P ) = 1 - {( 1 + P ( \frac{{K}_{0}^{′}}{{K}_{0}} ) )}^{\frac{1}{{K}_{0}^{′}}} \qquad Eq . ( 14 )$$

As a point of reference, when considering extreme pressures in earth and materials science, the third-order Birch-Murnaghan EOS (Equation 15 below) is used for bulk modulus characterization, which is derived from interatomic potentials.

$$P ( V ) = \frac{3 {K}_{0}}{2} [ {( \frac{{V}_{0}}{V} )}^{\frac{7}{3}} - {( \frac{{V}_{0}}{V} )}^{\frac{5}{3}} ] \{ 1 + \frac{3}{4} ( {K}_{0}^{′} - 4 ) [ {( \frac{{V}_{0}}{V} )}^{\frac{2}{3}} - 1 ] \} \qquad Eq . ( 15 )$$

However, because it is non-invertible (i.e., V cannot be described in terms of P), the Birch-Murnaghan EOS (Equation 15) is not useful for developing a powder compressibility equation. Invertibility is required for additivity with the other mechanisms in the lower pressure region captured by Equation 7. Additionally, the Murnaghan equation (Equation 12) can adequately describe elastic behavior under the pressures pertaining to uniaxial die compression of most powders (<2 gigapascal (GPa)). Hence, there are no tangible practical benefits from engaging the more complex Birch-Murnaghan equation (Equation 15).

It should be highlighted that K relates isostatic P and V, whereas powder compaction typically occurs under uniaxial P while the powder is confined within a die. The conditions are such that strain is only non-zero in a single dimension (axially), while stress is applied from both the axial and the radial direction via the die walls. In this instance, the measured value is the constrained elastic modulus (also known as the P-wave or longitudinal modulus), M, which is related to K according to Equation 16 below.

$$\frac{3 K ( 1 - ν )}{( 1 + v )} = M \qquad Eq . ( 16 )$$

Since M is related to K by a constant, an EOS describing V as a function of uniaxial P during die compression can be defined in a way analogous to K describing V as a function of isostatic P in Equation 7. Linear and nonlinear elasticity in the context of uniaxial die compression can, therefore, be described with Equations 17 and 18, respectively, below.

$${ε}_{E} ( P ) = 1 - {e}^{\frac{P}{M}} \qquad Eq . ( 17 )$$

$${ε}_{E} ( P ) = 1 - {( 1 + P ( \frac{{M}_{0}^{′}}{{M}_{0}} ) )}^{\frac{1}{{M}_{0}^{′}}} \qquad Eq . ( 18 )$$

Plastic deformation occurs at medium to high pressures, and is a major mechanism for the formation of a large bonding area for many powders. Of the available equations describing plastic deformation during powder compaction, the Heckel equation (Equation 5) is the most commonly used. The Heckel equation was derived on the assumption that porosity reduction via plastic deformation follows a first-order kinetics as:

$$\frac{d ε}{dP} = - \frac{ε}{{P}_{y}} \qquad Eq . ( 19 )$$

This separable differential equation can be solved for ε as a function of P to provide:

$$ε ( P ) = {ε}_{0} {e}^{- \frac{P}{{P}_{y}}} \qquad Eq . ( 20 )$$

Where ε₀ is the initial porosity at zero pressure. This can be transformed into the classical form of the Heckel equation (Equation 5) by taking the natural logarithm of both sides. Since this equation only concerns powder densification by the mechanism of plastic deformation, the actual e at zero pressure will be larger than the fitted ε₀ from this equation due to the porosity-eliminating effects of other low-pressure mechanisms, such as packing and rearrangement. Therefore, ε₀ can be redefined as ε_{y} to obtain Equation 21 below, which describes the porosity reduction as a function of pressure via plastic yield (ε_{Y}(P)) in Equation 7.

$${ε}_{Y} ( P ) = {ε}_{y} {e}^{- \frac{P}{{P}_{y}}} \qquad Eq . ( 21 )$$

Particles may undergo slippage, rotation, individual particle elastic deformation, and fragmentation in response to pressure during compaction, which results in significant powder densification. Analogous to the derivation of the Heckel equation, each mechanism may be modeled as a first-order pore-elimination process as below

$${\sum }_{i = 1}^{n} {ε}_{M , i} ( P ) = {\sum }_{i = 1}^{n} {ε}_{i} {e}^{- \frac{P}{{P}_{i}}} \qquad Eq . ( 22 )$$

Where εᵢ and Pᵢ is the porosity reduction and mean pressure attributed to i^{th} mechanism. This equation finds some support from the observation that tapped volume of a powder decreases exponentially with the number of taps.

Replacing terms in Equation 7 with Equations 18, 21, and 22 results in Equation 23 below.

$$ε ( P ) = 1 - {( 1 + P ( \frac{{M}_{0}^{′}}{{M}_{0}} ) )}^{\frac{1}{{M}_{0}^{′}}} + {ε}_{y} {e}^{- \frac{P}{{P}_{y}}} + {\sum }_{i = 1}^{n} {ε}_{i} {e}^{- \frac{P}{{P}_{i}}} \qquad Eq . ( 23 )$$

This may be simplified by grouping the plastic yield term in the summation term since it is also a first-order process, resulting in:

$$ε ( P ) = 1 - {( 1 + P ( \frac{{M}_{0}^{′}}{{M}_{0}} ) )}^{\frac{1}{{M}_{0}^{′}}} + {\sum }_{i = 1}^{n} {ε}_{i} {e}^{- \frac{P}{{P}_{i}}} \qquad Eq . ( 24 )$$

Since plastic deformation dominates in a pressure range higher than other lower pressure mechanisms, the highest Pᵢ value and corresponding εᵢ value in the summation term are equivalent to P_{y} and ε_{y}, respectively. Since

$${lim}_{P \to 0} ε ( P ) = {\sum }_{i = 1}^{n} {ε}_{i} , {ε}_{i}$$

is the porosity decrease and Pᵢ is related to the process half-life attributed to mechanism i, M₀ is the constrained modulus, and M′₀ is the first derivative of the constrained modulus.

In cases where linear elasticity adequately describes elastic deformation in the range of applied pressure, the elastic contribution term of Equation 24 can be replaced with the linear elasticity Equation 16, resulting in:

$$ε ( P ) = i - {e}^{\frac{P}{M}} + {\sum }_{i - 1}^{n} {ε}_{i} {e}^{- \frac{P}{{P}_{i}}} \qquad Eq . ( 25 )$$

In cases where even bulk elastic deformation is negligible due to relatively low applied pressures (typically for very hard materials), the elastic portion of Equation 25 may be omitted, resulting in:

$$ε ( P ) = {\sum }_{i = 1}^{n} {ε}_{i} {e}^{- \frac{P}{{P}_{i}}} \qquad Eq . ( 26 )$$

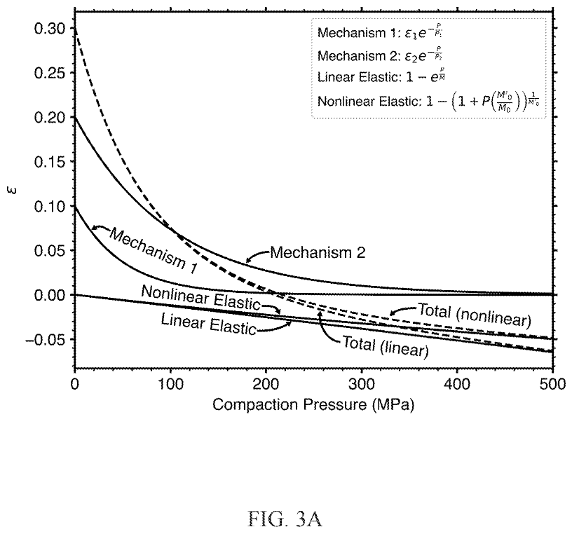

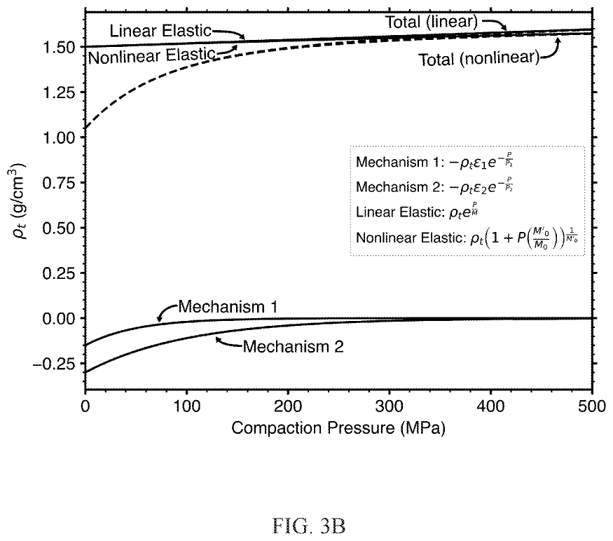

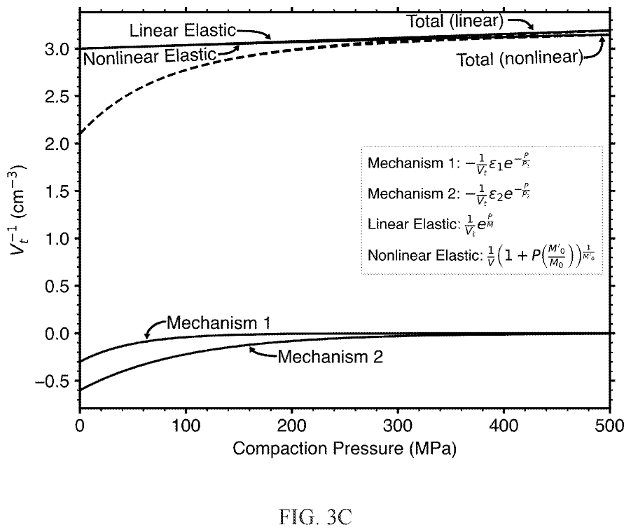

The contributions of each mechanism to the overall e as a function of P can be observed in the hypothetical example depicted in FIG. 3A that otherwise reflects ε as a function of P according to the developed additive models assuming two first-order mechanisms and hypothetical parameters given in Table 1 below.

TABLE 1

| | Hypothetical |
|---|---|
| Parameter | value |
| ρₜ (g/cm³) | 1.5 |
| Vₜ⁻¹ (cm⁻³) | 3.0 |
| M (GPa) | 8 |
| M′ | 10 |
| ε₁ | 0.1 |
| P₁ (MPa) | 50 |
| ε₂ | 0.2 |
| P₂ (MPa) | 100 |

One concern with using e is the reliance on material ρₜ. Small errors in ρₜ measurements can cause a large error in the measured material properties. Equation 24 can be converted to Equation 27 below, which describes ρ as a function of P by replacing ε with ρ. In Equation 27, ρₜ is a fitted parameter, where

$${lim}_{P \to 0} ρ ( P ) = {ρ}_{t} - {ρ}_{t} {\sum }_{i = 1}^{n} {ε}_{i} .$$

$$ρ ( P ) = {{ρ}_{t} ( 1 + P ( \frac{{M}_{0}^{′}}{{M}_{0}} ) )}^{\frac{1}{{M}_{0}^{′}}} - {ρ}_{t} {\sum }_{i = 1}^{n} {ε}_{i} {e}^{- \frac{P}{{P}_{i}}} \qquad Eq . ( 27 )$$

Equation 27 not only avoids errors from true density measurement but also allows for the extraction of ρₜ from compression data. This is advantageous since powder compaction may be performed using a very small amount of material (~0.1 g) and very quickly (<5 s).

If considering linear elasticity instead of nonlinear elasticity, Equation 27 is simplified as:

$$ρ ( P ) = {ρ}_{t} {e}^{\frac{P}{M}} - {ρ}_{t} {\sum }_{i = 1}^{n} {ε}_{i} {e}^{- \frac{P}{{P}_{i}}} \qquad Eq . ( 28 )$$

If no bulk elastic deformation contributes to the overall powder densification, Equation 28 simplifies to:

$$ρ ( P ) = {ρ}_{t} - {ρ}_{t} + {\sum }_{i = 1}^{n} {ε}_{i} {e}^{- \frac{P}{{P}_{i}}} \qquad Eq . ( 29 )$$

The contributions of each mechanism to the overall p as a function of P can be observed in the hypothetical example illustrated in FIG. 3B with the hypothetical parameters given in Table 1.

If a compact undergoes severe lamination upon ejection, an accurate weight may not be readily determined after ejection (provided the powder was not weighed before compression). In that case, methods of the present disclosure can use the definition of ρ (Equation 1) and ρₜ (Equation 3) to convert Equations 27-29 into Equations 30-32 below, using V⁻¹ as the dependent variable, where

$${lim}_{P \to 0} {V ( P )}^{- 1} = \frac{1}{{V}_{t}} - \frac{1}{{V}_{t}} {\sum }_{i = 1}^{n} {ε}_{i} .$$

$${V ( P )}^{- 1} = \frac{1}{{V}_{t}} {( 1 + P ( \frac{{M}_{0}^{′}}{{M}_{0}} ) )}^{\frac{1}{{M}_{0}^{′}}} - \frac{1}{{V}_{t}} {\sum }_{i = 1}^{n} {ε}_{i} {e}^{- \frac{P}{{P}_{i}}} \qquad Eq . ( 30 )$$

$$V {( P )}^{- 1} = \frac{1}{{V}_{t}} {e}^{\frac{P}{M}} - \frac{1}{{V}_{t}} {\sum }_{i = 1}^{n} {ε}_{i} {e}^{- \frac{P}{{P}_{i}}} \qquad Eq . ( 31 )$$

$${V ( P )}^{- 1} = \frac{1}{{V}_{t}} - \frac{1}{{V}_{t}} {\sum }_{i = 1}^{n} {ε}_{i} {e}^{- \frac{P}{{P}_{i}}} \qquad Eq . ( 32 )$$

As a point of reference, using V⁻¹ instead of V can avoid inverting the additive portion of the equation. Even though Vₜ will change from compact-to-compact (proportional to the mass of powder that is compressed), this equation still allows for the extraction of other parameters, like Pᵢ, εᵢ, M₀, and M′₀ from P−V⁻¹ data alone. The contributions of each mechanism to the overall V⁻¹ as a function of P can be observed in FIG. 3C with the hypothetical parameters given in Table 1.

It should be noted that ε, ρ, and V⁻¹ relationships are all equivalent descriptions of compressibility, but each has their own benefits and limitations when it comes to fitting compressibility data. ε versus P relationships (Equations 24-26) require one fewer fitted parameter (ρₜ), but require compact mass and an external ρₜ measurement, which can be unreliable for some materials. ρ versus P (Equations 27-29) still requires a compact mass, but adds ρₜ as a fitted parameter, thus allowing for a more flexible mathematical description of compressibility, a decreased reliance on external measurements, and detection of changes in ρₜ of the sample upon exposure to an environment with a different temperature or relative humidity. V⁻¹ versus P (Equations 30-32) does not require a compact mass, but also does not allow for determination of ρₜ and instead utilizes a mass-dependent Vₜ parameter.

Returning to FIG. 1, and as describe above, the assessment device 32 is configured or programmed to produce quantitative evaluation data or information directly or describing properties or parameters of the powder subjected to testing by the compression device 30, for example by operating one or more algorithms representative of one or more of Equations 24-32. As a point of reference, there are effectively three “sets” of equations: Equations 24, 27, and 30 implicate or are more appropriate for materials exhibiting nonlinear elasticity; Equations 25, 28, and 31 implicate or are more appropriate for materials exhibiting linear elasticity; and Equations 26, 29, and 32 implicate or are more appropriate for materials exhibiting no elasticity. Within each set, there are three subsets: equations determining or characterizing volume (Equations 30, 31, 32), equations determining or characterizing density (Equations 27, 28, 29), and equations determining or characterizing porosity (Equations 24, 25, 26), which are all interconvertible based on the definitions of porosity (ε), density (ρ), and volume (V). Alternatively or in addition, volume could be broken down further by considering that V=C(c,A)h+A*h for a compact, and h is dynamically measured by the compaction simulator (C(c,A) and A are constant); h is compact-shape-agnostic, and this approach can be beneficial for use with machines that do not have round-flat tooling.

The assessment device 32 can be programmed to automatically select a particular equation or set of equations based, for example, upon base properties of the material being evaluated and/or upon user prompts or input. The assessment device 32 and/or a user can choose which one to use within the subset (ε, ρ, or V) based on available information for the sample (e.g., sample mass, reliable true density, etc.). Where there is no information available regarding the elasticity characteristics of the sample being evaluated, various techniques can be employed. For example, the fitted true density from each “set” can be compared to a known true density value. Alternatively or in addition, additional compressions could be performed.

In yet other embodiments, the assessment device 32 can be programmed to operate one or more other algorithms or equations, for example that simplify one or more of the Equation sets or subsets identified above. For example, Equations 24, 25, and 26 can be replaced by Equation 33 below:

$$ε ( P ) = {ε}_{E} ( P ) + {\sum }_{i = 1}^{n} {ε}_{i} {e}^{- \frac{P}{{P}_{i}}} \qquad Eq . ( 33 )$$

Equations 27, 28, and 29 can be replaced by Equation 34 below:

$$ρ ( P ) = {ρ}_{t} ( 1 - {ε}_{E} ( P ) - {\sum }_{i = 1}^{n} {ε}_{i} {e}^{- \frac{P}{{P}_{i}}} ) \qquad Eq . ( 34 )$$

Equations 30, 31, and 32 can be replaced by Equation 35 below:

$${V ( P )}^{- 1} = \frac{1}{{V}_{t}} ( 1 - {ε}_{E} ( P ) - {\sum }_{i = 1}^{n} {ε}_{1} {e}^{- \frac{P}{{P}_{i}}} ) \qquad Eq . ( 35 )$$

$$Where {ε}_{E} ( P ) = 1 - {( 1 + P ( \frac{{M}_{0}^{′}}{{M}_{0}} ) )}^{\frac{1}{{M}_{0}^{′}}} \qquad ( Equation 18 )$$

for nonlinear elasticity,

$${ε}_{E} ( P ) = 1 - {e}^{\frac{P}{M}} \qquad ( Equation 17 )$$

for linear elasticity, and ε_{E}(P)=0 for no elasticity. All of these are inter-convertible.

In yet other embodiments, the assessment device 32 can be programmed to operate or apply a single, general equation (replacing Equations 24-32) as:

$$ε ( P ) = 1 - \frac{ρ ( P )}{{ρ}_{t}} = 1 - \frac{{V}_{t}}{V ( P )} = {ε}_{E} ( P ) + {\sum }_{i = 1}^{n} {ε}_{i} {e}^{- \frac{P}{{P}_{i}}} \qquad Eq . ( 36 )$$

$$Where {ε}_{E} ( P ) = 1 - {( 1 + P ( \frac{{M}_{0}^{′}}{{M}_{0}} ) )}^{\frac{1}{{M}_{0}^{′}}} \qquad ( Equation 18 )$$

for nonlinear elasticity,

$${ε}_{E} ( P ) = 1 - {e}^{\frac{P}{M}} \qquad ( Equation 17 )$$

for linear elasticity, and ε_{E}(P)=0 for no elasticity.

In the Examples section below, testing was performed to validate the ρ versus P relationships (Equations 27-29) of the present disclosure because it can be used to obtain ρₜ values by curve fitting. In this approach, the value of the fitted ρₜ can be compared to that independently measured to validate the equation obtained by fitting. Although several parameters are included in these equations, it is different from empirical fitting by introducing more terms. Instead, it is necessary to fully account for the complex process of uniaxial powder compression.

### EXAMPLES

Embodiments and advantages of features of the present disclosure are further illustrated by the following non-limiting examples. The particular materials and amounts thereof recited in these examples, as well as operating conditions and details, should not be construed to unduly limit the scope of the present disclosure.

Microcrystalline cellulose (MCC; Avicel® PH102, FMC Biopolymer, Philadelphia. PA), lactose monohydrate (LM; #316 Fastflo® NF, Foremost Farms, Clayton, WI), sodium chloride (NaCl; Macron Fine Chemicals, Allentown, PA), acetaminophen (APAP, Novacyl Pharmaceutical Co. Jiangsu, China) and dicalcium phosphate anhydrous (DCPA; Emcompress®, JRS Pharma, Patterson, NY) were used as received.

As part of the various testing and analyses described below, powder material samples were subjected to powder compaction. Compaction was performed using a Styl'One compaction simulator (Medelpharm, Beynost, France) using 6 mm round, flat-faced punches. Machine deformation was measured by compressing the punches against each other in an empty die up to the force of interest. The die walls and punch tips were lubricated three times with aerosolized magnesium stearate spray to reduce friction during ejection. Powder was loaded into the die by hand with a 10 mm fill depth. A single compression symmetrical profile operating at 1.7 mm/s was used to compress the powder between the two punches. Each compact was weighed on an analytical balance after ejection. In cases where intact compacts could not be obtained after ejection, the powder was pre-weighed on an analytical balance and was transferred to fill the die. The distance between punches and force data was exported to calculate compact volume, density, and porosity as a function of pressure by considering the punch tip area.

As part of the data analyses described below, nonlinear regression analysis was performed in Python (v3.10.8) using the Imfit (v1.0.3) package. To avoid the baseline noise before the punch makes contact with the powder, pressures below 1 megapascal (MPa) were excluded from fitting.

The ability to completely mathematically describe a compressibility curve for a powder gives significant insight into its properties and deformation behavior. There are two primary inputs required to fit the derived equations to ε, ρ, or V versus P data. First, the number of exponential mechanisms must be selected based on the best-fit to the data. Second, the type of material elastic response at the maximum compaction pressure (limited to about 1.4 GPa for purposes of this Examples Section) should be judiciously selected to accurately model the compaction process.

For the expressions of the present disclosure, the fitted model can, in some embodiments, benefit from user inputs that determine the quality and validity of the resultant parameters by fitting. An appropriate mathematic model for in-die powder compressibility depends on the number of deformation mechanism that contribute to volume reduction under pressure. In some embodiments, modeling with four exponentials may be sufficient for many materials. However, when more or fewer processes are involved in the compression of certain powders, the number of exponential terms needs to be adjusted accordingly. This can be done by comparing the fitted parameters. e.g., ρₜ, against expected values or by evaluating the fitted residuals, parameter significance, etc. Alternatively or in addition, evaluations can be performed using Akaike information criterion (AIC) or Bayesian information criteria (BIC) parameters.

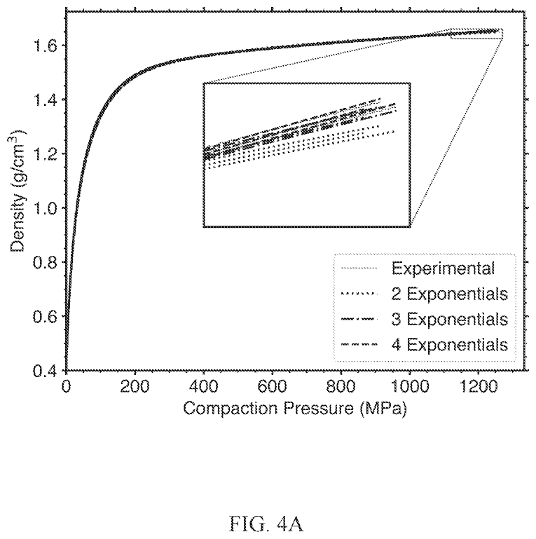

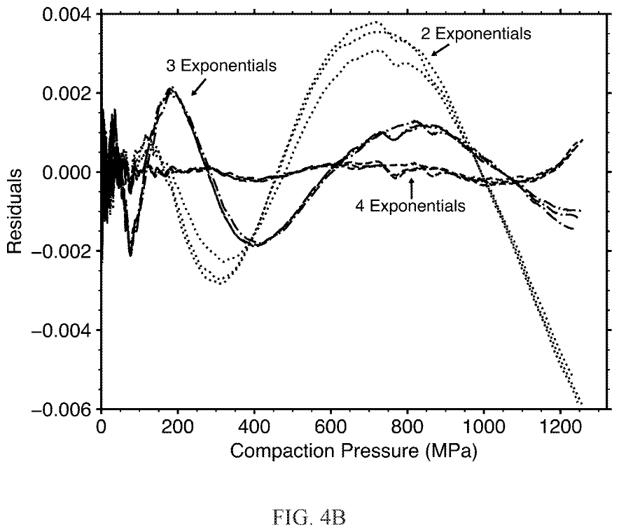

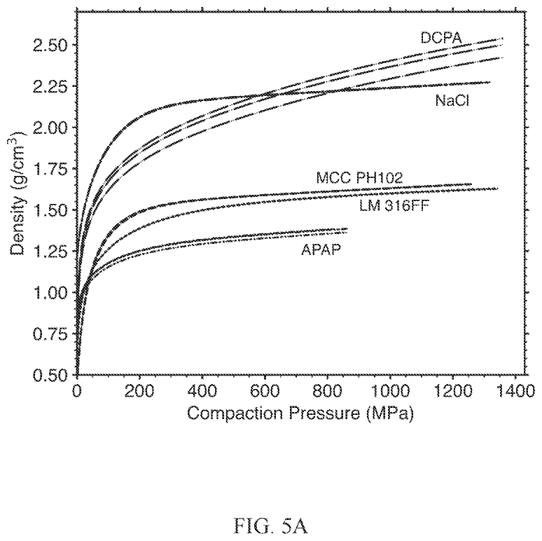

FIGS. 4A and 4B illustrate obtained fitting compression data for MCC PH102 using Equation 27 with 2, 3, and 4 exponential terms. FIG. 4A shows the fitting whereas FIG. 4B shows the residual of the fitting. Clearly, a function describing 4 processes following exponential models prior to bulk elastic deformation has the lowest residual values and has the best fit. The parameters of this fitting are also the most realistic, since the fitted M is closer to the measured K. The fitted parameters for MCC PH102 using Equation 27 with 2, 3, and 4 exponential terms examples are provided in Table 2 below. Values are represented as the average and standard deviation (in parenthesis) from triplicated results.

TABLE 2

| | 2 terms | 3 terms | 4 terms |
|---|---|---|---|
| ρₜ (g/cm³) | 1.4503 (0.0165) | 1.4863 (0.0053) | 1.5205 (0.0014) |
| M (GPa) | 3.0 (0.8) | 5.9 (0.6) | 12.1 (0.1) |
| M′ | 15.6 (1.1) | 11.7 (0.8) | 4.4 (0.1) |
| ε₁ | 0.480 (0.002) | 0.458 (0.001) | 0.33 (0.01) |
| P₁ (MPa) | 64.0 (1.0) | 70.6 (1.7) | 91.3 (4.1) |
| ε₂ | 0.233 (0.001) | 0.254 (0.0003) | 0.234 (0.004) |
| P₂ (MPa) | 13.3 (0.5) | 15.5 (0.5) | 34.5 (2.0) |
| ε₃ | — | 0.022 (0.002) | 0.158 (0.003) |
| P₃ (MPa) | — | 1.3 (0.1) | 11.8 (0.7) |
| ε₄ | — | — | 0.034 (0.001) |
| P₄ (MPa) | — | — | 0.62 (0.03) |

It may also be mentioned that the low-pressure cutoff (1 MPa) chosen for these Examples may influence the number of parameters that are significant during fitting. If a higher pressure cutoff is chosen, the data describing the lowest pressure process (ε₄ and P₄, in the case of MCC PH102) may be absent, which would affect the fitting. 1 MPa was chosen because it avoids the noise associated with the force measurement for a 6.0 mm punch while remaining relevant to powder consolidation. A punch with a larger surface area can capture low-pressure data at and below 1 MPa more accurately since more force will be required to attain such pressures, but the high-pressure plastic and elastic responses may not be attainable. With these Examples, the 6.0 mm punches provide sufficient resolution at low pressures (1 MPa and up) while keeping high pressures attainable, thus providing a balanced measuring apparatus.

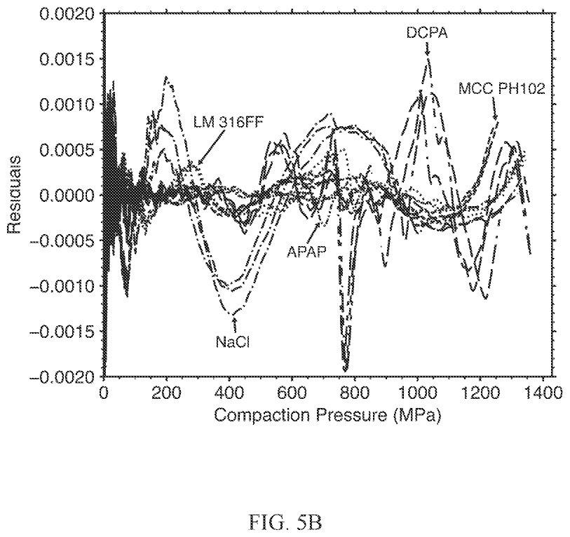

Theoretically, at high enough pressures, all materials will follow a nonlinear elastic response, e.g., MCC PH102. However, accessible pressures on a compaction simulator are limited to about 1.4 GPa to avoid bending of the punches. In this pressure range, elastic deformation is in the linear elasticity region for most pharmaceutical solids, e.g., LM 316FF. Hence, linear elasticity may be used as an initial estimate. Furthermore, for very hard materials, such as DCPA, constrained elasticity is negligible in this pressure region. Thus, contribution from elastic deformation may be omitted. Triplicated fitting curves for MCC PH102 (nonlinear elastic, four exponentials), LM 316FF (linear elastic, four exponentials), NaCl (linear elastic, two exponentials), APAP (linear elastic, five exponentials), and DCPA (zero elasticity, six exponentials) are shown in FIGS. 5A and 5B, that otherwise reflects the compressibility equations fitted to MCC PH102 (Equation 27), LM 316FF, NaCl, APAP (Equation 28), and DCPA (Equation 29) compaction data. FIG. 5A shows the fitting and FIG. 5B shows the residual of the fitting. The fitted parameters for MCC PH102, LM 316FF, NaCl, APAP, and DCPA compaction curves are summarized in Table 3 below. Values are represented as the average and standard deviation (in parenthesis) from triplicated results. The residuals of the fitting are very low and scattered around zero, indicating a good fit.

TABLE 3

| Material | MCC PH102 | LM 316FF | NaCl | APAP | DCPA |
|---|---|---|---|---|---|
| ρₜ (g/cm³) | 1.5205 (0.0014) | 1.5458 (0.0020) | 2.1339 (0.0018) | 1.3108 (0.0083) | 2.9427 (0.0659) |
| M (GPa) | 12.1 (0.1) | 24.6 (0.4) | 20.8 (0.1) | 15.4 (0.7) | — |
| M′ | 4.4 (0.1) | — | — | — | — |
| ε₁ | 0.33 (0.01) | 0.151 (0.003) | 0.358 (0.002) | 0.076 (0.005) | 0.406 (0.001) |
| P₁ (MPa) | 91.3 (4.1) | 316.3 (7.3) | 95.0 (0.8) | 335.1 (46.0) | 1410.7 (8.2) |
| ε₂ | 0.234 (0.004) | 0.188 (0.003) | 0.072 (0.001) | 0.108 (0.002) | 0.112 (0.001) |
| P₂ (MPa) | 34.5 (2.0) | 109.8 (1.3) | 14.8 (0.3) | 84.9 (8.0) | 131.7 (1.1) |
| ε₃ | 0.158 (0.003) | 0.148 (0.0004) | — | 0.094 (0.004) | 0.078 (0.0003) |
| P₃ (MPa) | 11.8 (0.7) | 23.14 (0.05) | — | 17.7 (2.1) | 35.9 (0.4) |
| ε₄ | 0.034 (0.001) | 0.128 (0.003) | — | 0.12 (0.008) | 0.077 (0.003) |
| P₄ (MPa) | 0.62 (0.03) | 4.66 (0.05) | — | 3.9 (0.5) | 11.3 (0.4) |
| ε₅ | — | — | — | 0.125 (0.016) | 0.072 (0.002) |
| P₅ (MPa) | — | — | — | 1.0 (0.1) | 3.8 (0.2) |
| ε₆ | — | — | — | — | 0.023 (0.002) |
| P₆ (MPa) | — | — | — | — | 0.79 (0.15) |

The selection of 4, 4, 2, 5 and 6 exponentials for MCC PH102, LM 316FF, NaCl, APAP, and DCPA, respectively, were chosen based on parameter significance after nonlinear regression. Interestingly, the two-exponential fitting for NaCl indicates only a single low-pressure process before plastic deformation. This indicates that NaCl is in a nearly ideal packing state after filling the die. This is consistent with larger particle size and more equidimensional shape compared to the other materials, which eliminated the need for some low-pressure mechanism like particle rearrangement. The large number of exponentials for DCPA may be a result of multiple fracture steps occurring at low pressures due to the brittle nature and rough surfaces of this material.

The elastic properties of MCC PH102, LM 316FF, NaCl, and APAP are quantified in Table 3. MCC PH102 shows nonlinear elasticity with a constrained modulus of 12.1 GPa and a modulus derivative of 4.4. LM 316FF, on the other hand, shows linear elasticity and has a constrained modulus of 24.6 GPa, which is about twice as large as MCC PH102. DCPA shows no identifiable elasticity since it is an extremely hard material. This M value for MCC PH102 (12.1 GPa) and APAP (15.4 GPa) is in reasonable agreement with the K measured by mercury porosimetry (10.2±1.0 GPa and 14.9±3.7 GPa, respectively), and the M for LM 316FF (24.6 GPa) is close to the K of lactose anhydrous (27.1±7.1 GPa). The M of NaCl (20.8 GPa) was found to be similar to K measured previously (24.0 GPa).

The elastic properties of powders have only recently been characterized using mercury porosimetry by Wunch et al., “A mathematical approach to consider solid compressibility in the compression of pharmaceutical powders”, Pharmaceutics, 11, 121 (2019), the entire teaching of which are incorporated herein by reference. Mercury porosimetry provides the appropriate environment to measure bulk modulus as it applies isostatic pressure. However, mercury porosimetry takes a significant amount of time (30-60 min) with the risk of mercury contamination. Additionally, the maximum attainable pressure is only around 400 MPa, which is not sufficient to induce a significant amount of elastic deformation for some materials like DCPA.

The systems and methods of the present disclosure utilizing in-die ρ versus P data from uniaxial powder compression allows for a swift (10-20 seconds) characterization of confined elastic deformation at pressures (up to 1.4 GPa in these examples) far above what can be achieved from standard mercury porosimetry. Additionally, this approach allows for the characterization of the nonlinear elastic behavior of highly deformable materials. Reasonable agreement between K determined from mercury porosimetry and M determined by uniaxial compression has been reported for materials, such as MCC and APAP. It should also be noted that the study of Wunch et al. accounts for linear elasticity using a simple linear dependence of pressure on sample volume, whereas some embodiments of the present disclosure describe the elastic deformation based on an explicit integration of Equation 8 and according to whether the material undergoes linear or nonlinear elastic behavior.

Being able to mathematically describe a material's elastic properties allows for the deconvolution of elasticity from other properties, like plasticity, which is discussed below. It can also allow for parity between in-die and out-of-die measurements, allowing for the prediction of out-of-die compressibility from in-die measurements, which are significantly more time- and materials-sparing. Finally, characterization of a material's elastic properties themselves can inform the decompression and ejection behavior of the compact, and may be predictive of capping and lamination behavior.

The P_{y} values for MCC PH102, LM 316FF, and DCPA are tabulated in Table 3 (labeled as the P₁ values). MCC PH102 is the most plastic, followed by NaCl, LM 316FF, APAP, and DCPA. These values are larger than those obtained through the conventional in-die Heckel analysis because the convolution of elasticity with plasticity in the conventional Heckel analysis artificially decreases the measured P_{y}. Since some methods of the present disclosure deconvolutes plasticity from elasticity, the extracted P_{y} is more accurate and is closer to that from the out-of-die Heckel analysis.

With the knowledge of contribution from elastic deformation in this analysis, plasticity may be separated from elasticity portion of in-die data. Additionally, more accurate results are expected when fitting using Equations 27-29 since potential errors in an externally measured ρₜ value are avoided. Furthermore, variability can be reduced further since the complete mathematical description of a compressibility curve eliminates the need to manually define a linear region, as was required in the standard Heckel analysis. Finally, this can aid in pinpointing the effect of environmental factors, like relative humidity, on material plasticity and compressibility, which was previously complicated by variable ρₜ measurements and researcher subjectiveness to the fitting pressure range.

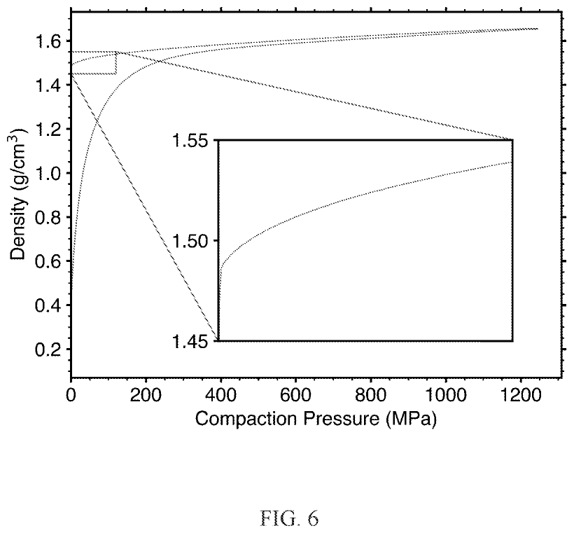

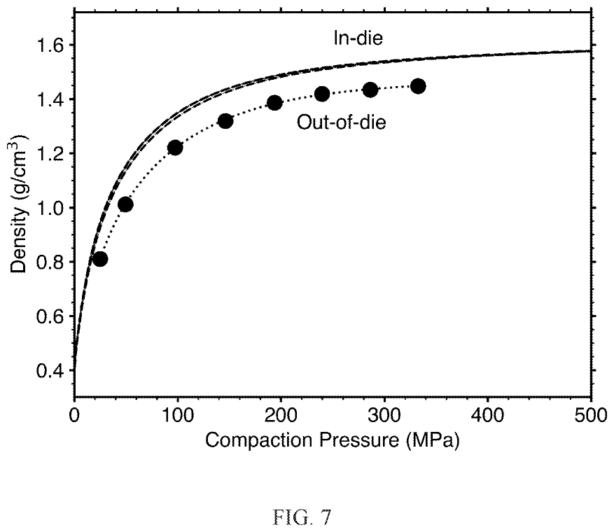

The fitted ρₜ of MCC PH102 (1.5202 grams per cubic centimeter (g/cm³)), LM 316FF (1.5458 g/cm³), NaCl (2.1339 g/cm³), APAP (1.3108 g/cm³), and DCPA (2.9427 g/cm³) (Table 3) is close to that obtained using helium pycnometry, indicating a reliable measure. The ~6% overestimation of the ρₜ of DCPA extracted via the compressibility equation may be due to undetectable elastic contribution under compression pressures employed in this work. Previous reports of MCC PH102's ρₜ being below 1.5 g/cm³ obtained through compression methods may be attributed to tablet relaxation after ejection, which result in a compact density below that of the ρₜ. Even before the MCC tablet was ejected from the die, tablet density at the end of decompression was already lower than the ρₜ as generally reflected in FIG. 6 that otherwise provides the compression and decompression curve for MCC PH102.

The intrinsic material property ρₜ has many implications for powder compression. Accurate characterization of ρₜ allows for an accurate determination of ε (Equation 2) and informs decisions made in many other powder processes. For example, during roller compaction in pharmaceutical manufacturing, compaction to a reasonable porosity is required to ensure that the resultant granules have sufficient compression properties.

As previously discussed, accurate ρₜ can be difficult to obtain using conventional methods or it takes a significant amount of time for accurate determination for some materials. This new method allows for the extraction of ρₜ using an in-die analysis of compressibility. Thus, an accurate ρₜ of an individual sample may be determined using just a small amount of powder (approx. 50-100 mg for a 6.0 mm die) in only seconds. Therefore, ρₜ can be quickly determined, which makes it suitable for routine determination of batch-to-batch variations in ρₜ.

The in-die and out-of-die compressibility differ due to decompression, ejection, and time-dependent recovery effects. Out-of-die compressibility may be considered a special case of in-die compressibility, where elastic deformation is negligible. Thus, the compressibility Equations 26 and 29 of the present disclosure can be used to describe out-of-die compressibility. Equation 32 cannot be used because m is not constant for all individual compacts.

Another challenge facing out-of-die compressibility characterization is the number of individual pressures used in the study. A standard out-of-die compressibility characterization of a typical pharmaceutical powder may typically utilize no more than 12 pressures between 25 and 350 MPa. This may not provide the granularity required to fully understand all of the mechanisms present during compression, especially at low pressures below 25 MPa where the powder is undergoing densification via rearrangement. The in-die analysis circumvents this problem by having access to dynamically collected P and ρ data throughout the course of a single compression cycle. Therefore, for out-of-die analyses, some embodiments of the present disclosure consider only a single low-pressure mechanism and plastic yield, resulting in Equation 37 below.

$${ρ}_{o} ( P ) = {ρ}_{t , o} - {ρ}_{t , o} {ε}_{1 , o} {e}^{- \frac{P}{{P}_{1 , o}}} - {ρ}_{t , o} {ε}_{2 , o} {e}^{- \frac{P}{{P}_{2 , o}}} \qquad Eq . ( 37 )$$

Equation 33 bears resemblance to the Cooper-Eaton equation (described in Cooper et al., “Compaction behavior of several ceramic powders”, J. Am. Ceram. Soc. 45, 97-101 (1962), the entire teachings of which are incorporated herein by reference), which similarly treats the reduction in volume as an additive function of a low- and high-pressure exponential association. Furthermore, by rearranging and redefining some parameters, Equation 33 is equivalent to the G-J equation that was used to describe the compressibility of titanium powders, and the Wünsch equation, which was used to characterize the ε versus P profiles of pharmaceutically relevant materials. This two-step model is more consistent with the classical descriptions of powder compressibility, which define just one low-pressure rearrangement mechanism followed by plastic deformation.

FIG. 7 illustrates a comparison of in- and out-of-die density versus compaction pressure plots for MCC PH102. In-die and out-of-die data of MCC PH102 fitted with corresponding compressibility equations are provided in Table 4. As a point of reference, the out-of-die data was obtained from Vreeman et al., “A powder tabletability equation”, Powder Technol., 408, 117709 (2022), the entire teachings of which are incorporated herein by reference. As shown, Equation 34 fits out-of-die data of MCC PH102 well (FIG. 5A). Compared to the in-die fitting for MCC PH102, the parameters for out-of-die data fitting are reasonably matched. The difference may be due to the convolution of low-pressure processes that cannot be modeled with the granularity of out-of-die data. As mentioned previously, the difference in true density can be attributed to recovery events during decompression. Thus, for a better reconciliation between in-die and out-of-die data, additional modeling of decompression events may be beneficial.

TABLE 4

| | In-die | Out-of-die |
|---|---|---|
| ρₜ (g/cm³) | 1.5205 (0.0014) | 1.4653 (0.0026) |
| M (GPa) | 12.1 (0.1) | — |
| M′ | 4.4 (0.1) | — |
| ε₁ | 0.33 (0.01) | 0.535 (0.003) |
| P₁ (MPa) | 91.3 (4.1) | 79.9 (3.3) |
| ε₂ | 0.234 (0.004) | 0.173 (0.003) |
| P₂ (MPa) | 34.5 (2.0) | 17.9 (7.0) |
| ε₃ | 0.158 (0.003) | — |
| P₃ (MPa) | 11.8 (0.7) | — |
| ε₄ | 0.034 (0.001) | — |
| P₄ (MPa) | 0.62 (0.03) | — |

The systems and methods of the present disclosure provide a marked improvement over previous designs. Powder compaction data is analyzed to extract several useful pieces of information that can be used, for example, to guide tablet formulation development. Only a small quantity of the material in question is required (e.g., on the order of less than one gram), and requisite powder compaction data can be obtained from a simple-to-operate in-die compression instrument or compaction stimulator. It was surprisingly found that an assumption of first-order kinetics of non-elastic mechanism is highly useful with the systems and methods of the present disclosure.

Although the present disclosure has been described with reference to preferred embodiments, workers skilled in the art will recognize that changes can be made in form and detail without departing from the spirit and scope of the present disclosure.

What is claimed is:

1. A method of assessing a powder material, the method comprising:
subjecting a quantity of the powder material to uniaxial compression;
collecting compaction data during the step of subjecting; and
fitting the collected data to a model.

2. The method of claim 1, wherein the step of fitting includes deriving one or more properties of the powder material.

3. The method of claim 2, wherein the one or more properties include includes at least one of:
fragmentation;
rearrangement;
plastic deformation;
nonlinear, linear, and zero elastic response;
elastic properties;
plastic properties;
true density; and
out-of-die compressibility.

4. The method of claim 3, further comprising:
outputting the derived one or more properties.

5. The method of claim 1, wherein the model is formatted to account for bulk elastic deformation.

6. The method of claim 1, wherein the step of subjecting includes subjecting less than 1 gram of the power material to uniaxial compression.

7. The method of claim 1, wherein the steps of subjecting the powder material to uniaxial compression and collecting compaction data is carried out over a time period of less than 5 seconds.

8. The method of claim 1, wherein the step of subjecting includes loading the quantity of the powder material into compression device with in-die measurement capabilities.

9. The method of claim 8, wherein the in-die compression device is a compaction stimulator.

10. A system for assessing a powder material, the system comprising:
a compression device operable to subject a quantity of the powder material to uniaxial compression and collect corresponding compaction data; and
an assessment device programmed to fit the collected data to a model.

11. The system of claim 10, wherein the assessment device is further programmed to derive one or more properties of the powder material based upon the fit.

12. The system of claim 11, wherein the one or more properties include includes at least one of:
fragmentation;
rearrangement;
plastic deformation;
nonlinear, linear, and zero elastic response;
elastic properties;
plastic properties;
true density; and
out-of-die compressibility.

13. The system of claim 12, wherein the assessment device includes a display, and further wherein the assessment device is programmed to output the derived one or more properties on the display.

14. The system of claim 10, wherein the model is formatted to account for bulk elastic deformation.

15. The system of claim 10, wherein the compression device is an in-die compression device.

16. The system of claim 15, wherein the in-die compression device is a compaction stimulator.

17. The system of claim 10, wherein the assessment device is programmed with at least one algorithm useful assessing a material exhibiting nonlinear elasticity, at least one algorithm useful for assessing a material exhibiting linear elasticity, and at least one algorithm useful for assessing a material exhibiting no elasticity.

18. The system of claim 17, wherein the assessment device is programmed with at least one algorithm useful for characterizing volume, at least one algorithm useful for characterizing density, and at least one algorithm useful for characterizing porosity.
